(19) United States
(12) Patent Application Publication
(10) Pub. No.: US 2025/0109378 A1
(43) Pub. Date: Apr. 3, 2025

(54) A POLYMER ASSEMBLY FOR GROWING CELLS

(71) Applicant: CATALYA, Liège (BE)

(72) Inventors: Christian GRANDFILS, Liège (BE); Chantal SEVRIN, Liège (BE)

(73) Assignee: CATALYA, Liège (BE)

(21) Appl. No.: 18/730,405

(22) PCT Filed: Mar. 22, 2023
(86) PCT No.: PCT/EP2023/057312

§ 371 Date: Jul. 19, 2024

(30) Foreign Application Priority Data
Mar. 22, 2022 (EP) 22163492.6

(51) Int. Cl. C12N 5/00; C12N 5/077

(52) U.S. Cl. CPC C12N 5/0075; C12N 5/0656; C12N 2531/00; C12N 2533/30; C12N 2533/74; C12N 2537/00; C12N 2539/10

(57) ABSTRACT

A coated surface with a polymer substantially consisting of a succession of monomers according to the formula (I) and its use for growing cells.

### TECHNICAL FIELD

The present invention relates to a polymer for use in the coating of surfaces, useful for growing cells.

### PRIOR ART

Animal cell cultures are daily used in routine either for various in vitro applications amongst other for R & D activities, diagnostic applications, cytotoxicity and pharmacological assays. Animals cells are also broadly used in the pharmaceutical industries in order to produce biopharmaceutical active ingredients, such as recombinant proteins, vaccines, nucleic acids (DNA, mRNA, . . . ), exosomes, viral vectors. Today animal cells are also conceivable as an end-therapeutic product in the frame of cell therapy and tissue engineering for organ repair or even organ replacement.

Most of these animal cells are so-called adherent animal cells, because, when cultured in vitro, their short-term survival is strictly dependent on their ability to adhere quickly to a solid surface, which has to be tailored to promote cell attachment, then cell proliferation, followed afterwards by cells differentiation. During these steps, cells undergo morphological changes arising from their passive deformation, but also the reorganization of their cytoskeleton. In vivo, but also in vitro this cell adhesion process is essential for cell communication and regulation and is crucial for cell development and maintenance.

This dependence of the survival of animal cells to their adhesion to a solid surface is imposing to develop enough surface accessible to the cells when trying to amplify them to large-scale level. Indeed, grossly taking into account that the typical mean surface area required per cell is close to 350 μm², a total area of 0.2 m² is required to produce a therapeutic dose of stem cells for cell therapy (i.e. 500 million of cells) and that a total area of 2000 m² is typically needed to cultivate animal cells in a 2000 L bioreactor for vaccine production.

At the laboratory level, cells are cultivated on 2D surfaces using typically multiwell plates or T-Flasks whilst the biopharmaceutical industry is adopting 3D cell culture support and doing cell cultivation in bioreactors. Both 2D or 3D surface material needs to be optimized in order to promote cell adhesion and proliferation.

In vivo, cell adhesion to either other cells or to the extracellular matrix is mediated by proteins, such as integrins. These transmembrane proteins act as transducer to the cell cytoskeleton extensors and actin filaments promoting the formation of focal adhesion complex.

In the case of in vitro cell culture, no specific biological ligands are applied on the surface of the materials used. Cell adhesion mostly depends of lesser specific interactions & weak bonds, which can rely on hydrophobic or van der Waals interactions. However, animal cells possess a negative surface-charge at physiological pH. Interestingly, cells can however be cultivated on both positive (DEAE-ion exchanger) or negatively charged surfaces. Interestingly, the experience has demonstrated that it is not the polarity of the charges, but the surface charge density on the culture surface which is key to the cell adhesion. Critical values need to be achieved to promote cell adhesion and proliferation. This optimal value corresponds to a discrete range of surface charges density which is for negatively charged surfaces within the range of 2-10 charges/nm² and about 600 charges/nm² for positively charged surfaces. Above these values, the cell growth is inhibited or cells are dying, and below those values, cell adhesion is very poor. This critical surface charge density is also affected by the interaction between proteins or polymers contained in the culture medium and which are adsorbed on the surface, and thus exposed to the cells.

The material most widely used for in vitro cell culture for 2D animal cell culture is polystyrene. This polymer is very hydrophobic and devoid from any charges. Without any treatment, polystyrene does not promote animal cell adhesion and is therefore only suitable for the culture of non-adherent cells. In contrast, when polystyrene surfaces are chemically or physically treated to introduce ionic groups, typically anionic groups present on tissue culture polystyrene (TCPS), its surface is rapidly covered by a thin layer of proteins upon incubation with serum-containing culture medium, allowing the deposition of extracellular matrix, which favour cell adhesion.

If TCPS can be used for several animal cell lines, different strategies have been reported to improve the adhesion of fibroblasts and osteoblasts in vitro and to better control this. Either functional non-specific groups have been anchored on the surface of the polystyrene in order to increase at least their hydrophilicity and their surface charge density, either ligands recognizing specific cell integrins have been also chemically attached to the material surface. Different synthetic or natural macromolecules such as poly-L-lysine or proteins characteristics of the extracellular matrix, i.e. fibronectin, vitronectin, collagen and laminin, have been also either physically adsorbed or chemically grafted on polymer surface.

However, when reaching cell confluence in vitro, cells have to be detached from the solid surface in order to ensure a good viability ration. This harvesting process, which should be repeated on a regular basis, every 7 to 10 days, sometimes more frequently, is called a “cell passage”. Until now this cell detachment, both at laboratory scale and the industrial scale, request to use proteolytic enzymes to hydrolyse the integrins or part of their extra-cellular fragments allowing to release eukaryotic cells from the surface. This harvesting process is suffering from several drawbacks, chief amongst which are the cost of the enzyme treatment and the risk of cell apoptosis by anoikis. Therefore, this enzymatic treatment should be kept to a minimum impact, with a rapid neutralization of the proteolytic enzymes by addition of inhibiting proteins. For in vivo applications, the enzymatic contamination of stem cells can raise safety concerns. This enzymatic treatment, alone or combined with calcium chelating agents, such as EDTA, has been also reported as poorly efficient, especially when dealing with stem cell detachment from various microcarriers.

Alternative strategies have been evaluated to replace enzymatic treatment by alternative methodologies which could rely on simple physico-chemical triggering events more compatible with eucaryotic cell viability. Different stimuli could be used in order to change the physico-chemical behaviour of polymers coated to a surface and potentially to control cell adhesion more easily. Amongst several techniques described, one can mention: change in temperature, application of an electric field, local dissipation of energy under the form of light, ultrasound, magnetic fields or the application of mechanical agitations.

The thermal-inducible polymers, N-lsopropylacrylamide (pNIPAM) is the most studied thermal-inducible polymers in its category, with a phase transition around 32° C., i.e. close to the physiological conditions. Above the LCST (Lower Critical Solution Temperature) of 32° C. the hydrogen bonds responsible of the solvation of pNIPAM are essentially broken, inducing a change of its conformation to a collapsed globular and hydrophobic form favourable to cell adhesion. Below 32° C., pNIPAM have hydrated, hydrophilic and mobile chains which are ensuring steric repulsion forces inhibiting protein adsorption promoting animal cell detachment. However, in practice, the efficiency and kinetics of this non enzymatic cell detachment process has been disclosed to be low and the material commercially available today are expensive due to the technical challenges related to the grafting of pNIPAM on polystyrene.

The pH is an important environmental parameter for biomedical applications, because pH changes occur in many specific or pathological compartments. The key element for pH responsive polymers is the presence of ionisable, weak acidic or basic moieties that attach to a hydrophobic backbone, such as polyelectrolytes. Upon ionization, the electrostatic repulsions of the generated charges (anions or cations) cause a dramatic extension of coiled chains. The ionization of the pendant acidic or basic groups on polyelectrolytes can be partial, due to the electrostatic effect from other adjacent ionized groups. Accordingly small changes in pH can induce, very abruptly, phase transition in pH responsive polymers. Some well-known pH responsive polymers are chitosan, albumin, gelatine, poly(acrylic acid), poly(methacrylic acid-g-ethylene glycol), poly(ethylene imine) (PEI) or poly(lysine) (PL). However no smart surface able to control cell adhesion and cell detachment have been demonstrated successful until now adopting those list of polymers

Hence, unfortunately, the prior art's methods are not efficient enough.

### BRIEF DESCRIPTION OF THE INVENTION

It is the object of the invention to improve the in vitro culture of animal cells by enhancing both (i) the cell adhesion process and (ii) the cell detachment, when needed.

According to the invention, this object is solved by providing a simple, rapid and cost-effective physical coating of a cell culture substrate by a polymer (herein after referred to as PDMAEMA) consisting essentially of a succession of monomers according to the formula I:

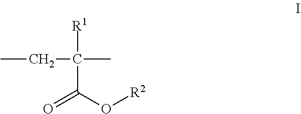

wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group.

This poly(N,N-diakylamino ethylmethacrylate) PDMAEMA, when coated on the surface of materials used in cell culture, promotes either cell adhesion or cell detachment through a moderate and quick change in pH of the culture medium. This is due to very important change in the polymer charge density, being mostly positive at pH 7.4 and neutral close to pH 8.0.

A first aspect of this invention is a method to grow animal cells comprise the steps of:
selecting a surface
coating the said surface with a polymer substantially consisting of a succession of monomers according to the formula I
growing these animal cells on this coated surface for a ((pre)determined) period of time so that these animal cells adhere to this coated surface.
   Advantageously, this method further comprises a (final) step of detaching and dissociating the animal cells from the surface, preferably without any protease treatment, for instance, by a pH increase and/or addition of chelator of bivalent ions or/and as competitor of the polymer coated to the surface. The preferred competitors are low molecular cationic (or zwitterionic) molecules (hence a molecular weight of less than 500 Da, preferably less than 300 Da), preferably betaine, spermidine or spermine.

A second aspect of this invention relies on the same polymer (poly(N,N-diakylamino ethylmethacrylate) PDMAEMA), applied on dissolvable microcarriers, preferably comprising, or consisting essentially of, alginate.

### BRIEF DESCRIPTION OF THE DRAWINGS

FIG. 1. Microscopic observations of L929 fibroblasts 4 or 7 days after culture on 24 multiwell made of TCPS or PS.

FIG. 2. Evolution of the fluorescence signal intensity (expressed in brightness intensity measured by image data analysis) in function of the concentration of the polymer solution incubated in each TCPS multiwell.

FIG. 3. Evolution of the polymer adsorbed per well (μg) in function of the polymer amount added per 24 multiwell. The results have been also expressed in terms of % of polymer adsorbed per well.

FIG. 4. Evolution of the polymer released in the culture medium at 37° C. with time in function of the polymer originally applied per well. From day 0 to day 3, the culture medium was buffered at pH 7.4 and was containing FBS (10%). After 3 days, this culture medium has been replaced by DMEM buffered at pH 8.0.

FIG. 5. Microscopic observations of L929 fibroblasts 4 days after culture on 24 multiwell made of TCPS or PS without or with coating PDMAEMA solutions of 1 or 10 mg/mL

FIG. 6. Microscopic observations of L929 fibroblasts 7 days after culture on 24 multiwell made of TCPS or PS without or with coating PDMAEMA solutions of 1 or 10 mg/mL

FIG. 7. Microscopic observations of L929 fibroblasts 1, 5 and 7 days after culture on 24 multiwell made of TCPS without or with coating PDMAEMA solutions of concentration of 0; 0.1; 0.25; 0.75 or 1 mg/mL.

FIG. 8. Microscopic observations of L929 fibroblasts cultured for 7 days on 24 multiwell made of TCPS, without or with coating PDMAEMA (1 mg/mL) or on PS coated with PDMAEMA (1 mg/mL). These observations have been taken before and after cell harvesting either by trypsinisation or incubation at pH 8.0.

FIG. 9. MTT (Cell viability assay which relies on the reduction of the tetrazolium dye MTT, i.e. 3-(4,5-dimethylthiazol-2-yl)-2,5-diphenyltetrazolium bromide) counting of fibroblasts L929 after culture for 7 days on 24 multiwell made of either TCPS or PS, without or with coating PDMAEMA solutions (1 mg/mL). Before cell counting the cells have been harvested either by trypsinisation or incubation at pH 8.0.

FIG. 10. Microscopic observations of L929 fibroblasts cultured for 5 days on 24 multiwell made of TCPS or TCPS coated with PDMAEMA (1 mg/mL) after their harvesting by either trypsinisation or incubation at pH 8.0. The cells have been afterwards seeded on 96 multiwells TCPS for 2 h before observing under microscopy and carrying out the MTT.

FIG. 11. MTT counting of fibroblasts L929 after culture for 5 or 7 days on 24 multiwell made of either TCPS, without or with coating PDMAEMA solutions (0.1; 0.25; 0.75 or 1 mg/mL). Before cell counting the cells have been harvested either by trypsinisation or incubation at pH 8.0.

FIG. 12. Microscopic observations of Vero cells after thawing, seeding and cultured for 7 days on 12 multiwell made of TCPS or TCPS coated with PDMAEMA (1 mg/mL).

FIG. 13. Epifluorescence observation of alginate microcarriers coated with raising concentration of a fluorescent PDMAEMA solution (batch CS086) in water.

FIG. 14. Comparison of the epifluorescence signal of alginate microcarriers coated with a fluorescent PDMAEMA dissolved in water at 3 concentrations before, and after autoclaving.

FIG. 15. Epifluorescence observation of alginate microcarriers coated with raising concentrations (0.01 to 1 mg/mL) of a fluorescent PDMAEMA incubated for 1 day in NaCl (9 g/L), followed by 3 days in DMEM with FBS (10%) at 37° C.

FIG. 16. Epifluorescence (16A) and normal light (16B) micrographies of alginate microcarriers coated with fluorescent PDMAEMA after their incubation in PBS for 90 min at RT in view to promote their dissolution.

FIG. 17. Dynamic Light Scattering and fluorescent analysis of the dissolution medium after dissolution of alginate microcarriers coated with fluorescent PDMAEMA in an EDTA solution (2.5 mM) performed at room temperature for 15 min.

FIG. 18. Microscopic observation of L929 fibroblasts cultured on the surface of PDMAEMA coated alginate microcarriers 3 days post seeding.

FIG. 19. Microscopic observation of the alginate microcarriers after chitosan coating and incubation in EDTA (5 mM).

### DETAILED DESCRIPTION OF THE INVENTION

The inventors have formulated a novel approach of surface functionalization of material, relying on the simple, rapid and cost-effective physical coating of poly(N,N-diakylamino ethylmethacrylates), PDMAEMA, on the surface of materials used in cell culture. Through a moderate and quick change in pH of the culture medium, this polymer promotes either cell adhesion or cell detachment, due to its very important change in charge density, being mostly positive at pH 7.4 and neutral close to pH 8.0.

A first aspect of the present invention is thus a method to grow animal cells, preferably mammalian cells, comprising the steps of:
selecting a surface,
coating the said surface with a polymer substantially consisting of a succession of monomers according to the formula I

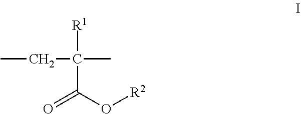

and
growing these animal (mammalian and/or stem) cells on this coated surface for a (pre(determined)) period of time so that these animal (mammalian and/or stem) cells adhere to this coated surface,
wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group;
wherein this polymer has a mean pKa between 6.0 and less than 7.5 and a charge density at pH 7.0 between 20 and 50% (number of the positively charged monomers:total number of monomers).

This allows the coating of surface(s) with a polymer having a defined density of positive charges.

Preferably, the method of the present invention comprises the preliminary step of determining the optimal charge density of the polymer substantially consisting of a succession of monomers according to the formula I. Advantageously, several surfaces are coated with a diversity of the polymer substantially consisting of a succession of monomers according to the formula I, before application of the culture medium for a predetermined period of time and application of the animal (human and/or stem) cells and the most suitable polymer is selected. Preferably, structures (III) to (XIV) here below are incorporated to a certain extent to the polymer substantially consisting of a succession of monomers according to the formula I, which will fine-tune the charge density.

This allows to identify the ideal polymer, depending (i) on the surface, (ii) on the culture condition and (iii) on the type of cells.

Preferably this animal and/or mammalian and/or stem cells are not human embryonic stem cells.

The surface can be a 2D surface or a 3D surface (such as alginate beads; for instance with a diameter of about 150-500 μm), preferably between 200 and 300 μm, which is advantageous for large-scale production.

In the present invention, negatively-charged surfaces to be coated by the polymer of the present invention are preferred. In the context of the present invention, the terminology “negatively charged surfaces” means a surface (an uncoated surface) having a density of negative charges higher than 10/nm², preferably higher than 50/nm², more preferably higher than 100/nm² and/or more negative charges than positive charges per surface unit (uncoated; hence without taking into account the polymers to be adsorbed on the surface, including the polymer of the present invention, or proteins, polycations, polylysine, chitosan . . . , added through the culture medium or added as a coating).

However, the polymer of the present invention can also be advantageous for neutral or positively charged surfaces, either the surface per se, or a surface further coated by a positively-charged substance (subsequently coated by the polymer according to the present invention).

The method further comprises the advantageous step of detaching the grown animal (mammalian and/or stem) cells upon increasing the pH of the medium to a pH comprised between 7.8 and 8.5 (preferably between 7.9 and 8.2, such as about 8.0; the pH is preferably the highest possible that does not detrimentally affect cell viability).

Advantageously, the detachment is achieved in synergy with the addition of calcium chelators, such as (sterile) EDTA, for instance between 0.5 and 5 mM. The addition of calcium chelators allows a gentle detachment of the cells, without relying on proteases or on treatments that can be toxic for the cells, such as higher pH values.

Alternatively, or in a complementary way cationic molecules of low molecular weight can be added in order to dissociate polyelectrolyte complexes made between the coating polycation and proteins/animal cells adhering to the solid substrate. As example of cationic molecules can be mentioned, betaine, tris(hydroxymethyl)aminomethane, putrescine, spermidine or spermine.

Advantageously, these methods allow a gentle detachment of the cells and can support cell dissociation, without relying on proteases or on treatments that can be toxic for the cells, such as higher pH values.

Preferably, the R¹ is a methyl group.

Preferably, this polymer (used in the method of the present invention or present in the composition of the present invention) is comprising more than 80%, preferably more than 90% and more preferably more than 95% of the monomers according to the formula I, such as between 95% and 99% (number of the monomers according to the formula I:total number of the monomers).

In other words, preferably, in the context of the present invention (method and composition), the wording “substantially consisting of a succession of monomers according to the formula I” means that more than 60%, preferably more than 65%, more than 70%, more than 75% or even more than 80% of the monomers are those according to the formula I (monomers according to the formula I:total number of the monomers, including those forming the alpha and omega extremities), preferably more than 85, 90, 91, 92, 93, 94, 95, 96, 97, 98% are those according to the formula I.

The remaining monomers, herein after referred to as “complementary monomers” are preferably of an acrylate structure with no protonable amine, such as methyl methacrylate, ethyl methacrylate, propyl methacrylate or butyl methacrylate. This allows to fine-tune the density of the positive charges (at pH between 7.0 and 7.5) of the polymer. These complementary monomers will thus be incorporated, possibly in the form of a copolymer with the monomer according to the formula (I).

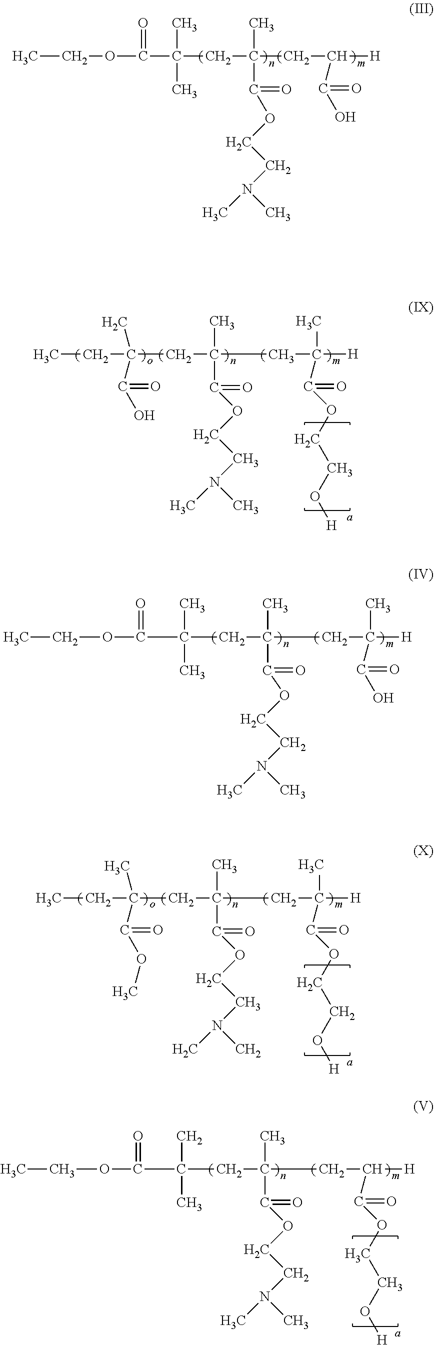

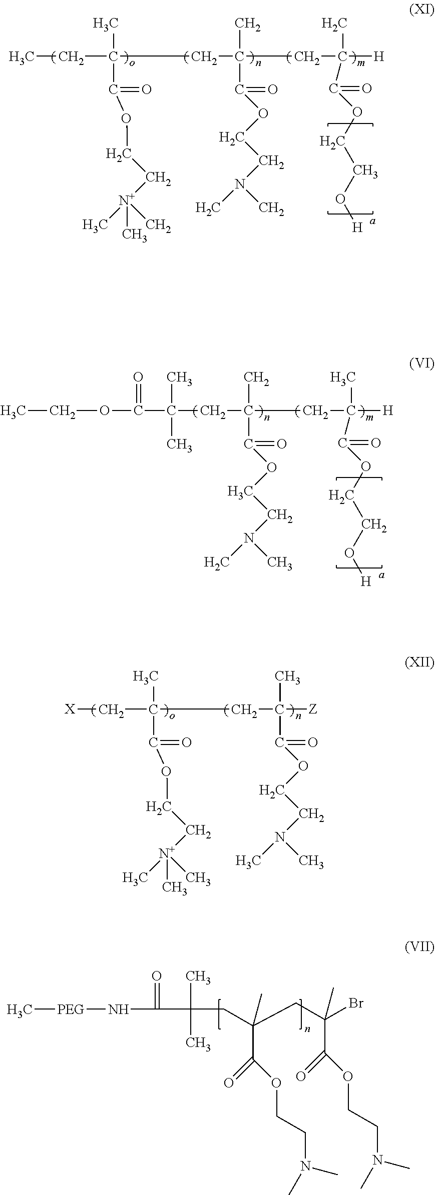

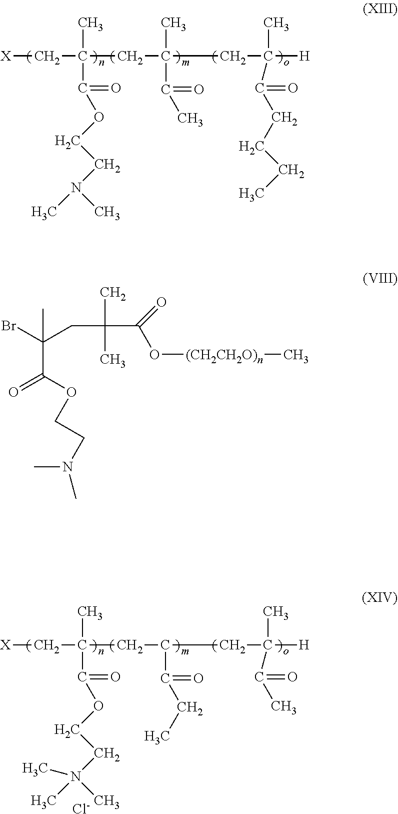

Examples of copolymeric (and terpolymeric) structures which could be used include poly[2-(dimethylamino)ethyl methacrylate)-co-acrylic acid] (III), poly[2-(dimethylamino)ethyl methacrylate)-co-methacrylic acid] (IV), poly[2-(dimethylamino) ethyl methacrylate)-co-poly(ethylene glycol) a-methyl ether, co-acrylate] (V), poly[2-(dimethylamino) ethyl methacrylate)-co-poly (ethylene glycol) a-methyl ether, co-methacrylate] (VI), poly[2-(dimethylamino) ethyl methacrylate)-co-poly(ethylene glycol)] (VII), poly[2-(dimethylamino) ethyl methacrylate)-co-poly(ethylene glycol)] (VIII), poly[methacrylic acid-co-2-(dimethylamino) ethyl methacrylate)-co-poly(ethylene glycol) a-methyl ether, co-methacrylate] (IX), poly[methyl methacrylate-co-2-(dimethylamino) ethyl methacrylate)-co-poly(ethylene glycol) a-methyl ether, w-methacrylate] (X), poly[trimethylamino) ethyl methacrylate-co-2-(dimethylamino) ethyl methacrylate)-co-poly(ethylene glycol) a-methyl ether, co-methacrylate] (XI), poly[trimethylamino) ethyl methacrylate-co-2-(dimethylamino) ethyl methacrylate] (XII), poly[2-(dimethylamino) ethyl methacrylate-co-methyl methacrylate-co-butyl methacrylate] of formula (XIII); and poly[2-(trimethylamine) ethyl methacrylate chloride-co-ethyl acrylate-co-methyl methacrylate] of formula (XIV).

The polymer organization can be adapted depending on the molecule to load: either by random polymerization, or by designing segments consisting of a succession of the monomers according to the Formula I and a succession of the complementary monomers. This allows, for instance, to generate more hydrophobic regions (segments) and more hydrophilic regions (segments) within the same polymer molecule.

The complementary monomers can, possibly, be branched by other oligomeric segments, provided that the polymer keeps its advantageous properties.

The nature, abundance and method of incorporation of the complementary monomers is chosen according to physico-chemical properties needed.

The selection and architecture of the complementary monomers is, of course, more important when they are incorporated in higher amounts, such as when more than 20%, more than 25% more than 30% or even close to 40% of the monomers are complementary monomers: a proportion of at least 60% of the monomers corresponding to the formula I confers the basic structure and physico-chemical properties, and the complementary monomers fine-tune these properties.

The polymerization process can be performed by several methods. Preferably anionic polymerization or free-radical polymerization are used.

Convenient synthesis protocols are indeed based on free-radical polymerization.

ATRP (Atom Transfer Radical Polymerization) is a preferred method of synthesis, especially when there is no need of a specific architecture of the complementary monomers.

RAFT (Reversible Addition Fragmentation Chain Transfer) polymerization has also been successfully found useful for a better control of the polymer organization.

Preferably, in the method of the present invention, the polymer harbors a charge density at pH 7.0 between 25 and 40%, preferably between 30 and 35% (number of the positively charged monomers:total number of monomers). Conversely, preferably, the polymer of the present invention harbors less than 10% of negative charge density at pH 7.0, preferably less than 5%, more preferably less than 2% (number of the negatively charged monomers:total number of monomers).

Preferably, in the method of the present invention, the polymer is coated on the surface upon incubation at a concentration between 0.1 mg/ml and 1.0 mg/ml, preferably 0.15 mg/ml and 0.5 mg/ml, more preferably between 0.2 mg/ml and 0.3 mg/ml.

Preferably, the polymer is according to the formula II

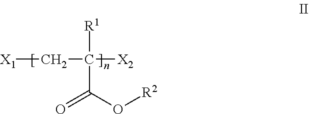

wherein X₁ and X₂ represent the alpha and omega end groups of the said polymer;
wherein the said X₁ and X₂ independently represent a hydrogen atom, a hydroxyl group, an ethyl isobutyrate group, an alkyl group, a halogen group, a carboxylic acid group, an amino group, a methoxy group or an ethoxy group and wherein n is the number of the repetitive monomer units according to the formula I.

A related aspect of the present invention is a composition comprising a surface coated with a polymer substantially consisting of a succession of monomers according to the formula I

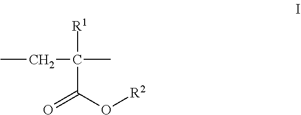

wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group.

Preferably, this said polymer has a mean pKa between 6.0 and 7.5 and/or a charge density at pH 7.0 between 20 and 50% (number of the positively charged monomers:total number of monomers).

This allows to adapt in view of the nature of the surface to coat and/or the cell to adhere.

The surface (before coating) is preferably negatively charged, preferably selected from the group of plasma-treated polystyrene, biopolymer, glass and alginate.

Advantageously, alginate polymers, coated with the polymer of the present invention allows dissolvable carriers for cell culture.

Hence, another related aspect of the present invention is a composition of dissolvable particulate carriers for cell culture, the carriers comprising a core being dissolvable and tailored to be insoluble in classical cell culture medium in view to serve as a stable surface to promote cell adhesion and proliferation. Typically the core of these microcarriers is made from an anionic polysaccharide, such as sodium alginate or their derivatives, which is easily physically reticulated with calcium ions upon formation of particles. Due to the high anionic surface density of alginate macromolecules, animal cells do not adhere on it. However upon coating with the polycationic polymers disclosed above, the Zeta potential of the dissolvable microcarriers can be reduced, and possibly reversed to promote animal cell adhesion. Advantageously after animal cell culture, these functionalized microcarriers are easily redissolved in smooth conditions to release efficiently the cells in the culture medium. Accordingly cells are easily recovered and purified from the resulting solution.

Advantageously, these dissolvable carriers have a particulate form and are kept in suspension, allowing a sharp increase of the cell surface.

Preferred particulate carriers are substantially spherical.

The controlled dissolvable microcarriers can be (entirely) dissolved by adding low molecular compounds such as phosphate anions (as typically included in physiological buffer saline; PBS) or/and EDTA (0.5 to 5 mM). These physiological medium are indeed able to compete and displace easily and quickly calcium ions bounded to alginate.

This approach is more advantageous than stable microcarriers such as Cytodex or Plastic microcarriers, those particles are known to generate non degradable or/and biocompatible particle fragments, due to the agitation requested to maintain them in suspension during cell cultivation, with similar size than animal cells: these particles are therefore very difficult to separate from the cells.

Preferably the polymer is comprising more than 80%, preferably more than 90% and more preferably more than 95% of the monomers according to the formula I, such as between 95% and 99% (number of the monomers according to the formula I:total number of the monomers), and/or wherein R1 is a methyl group.

Preferably, the polymer harbors a charge density at pH 7.0 between 25 and 40%, preferably between 30 and 35% (number of the positively charged monomers:total number of monomers), and/or harbors less than 10% of negative charge density at pH 7.0, preferably less than 5%, more preferably less than 2% (number of the negatively charged monomers:total number of monomers).

Advantageously, the polymer has been coated on the surface upon incubation at a concentration between 0.0001 mg/ml and 1.0 mg/ml, preferably 0.01 mg/ml and 0.5 mg/ml, more preferably between 0.05 mg/ml and 0.3 mg/ml, such as between 0.1 and 0.3 mg/ml.

Preferably, this composition is further comprising animal cells, preferably mammalian cells, adhered on the polymer coated on the surface, wherein these animal and/or mammalian and/or stem cells are preferably not human embryonic stem cells.

Advantageously, the animal and/or mammalian cells are adhered or inoculated at a known (subconfluent) density.

Another related aspect of the present invention is the use of the composition of the present invention or of the polymer consisting essentially of the formula I for an in vitro culture of animal cells, preferably of mammalian cells, including stem cells of different mammalian origin, including human type (not human embryonic).

Another related aspect of the present invention is a method to produce a 3D support for cell culture comprising the step of:
selecting or obtaining alginate beads;
coating these beads with the polymer described here above (e.g. comprising more than 95% of the monomers according to the Formula 1), preferably by suspending the beads in a solution comprising the polymer described here above (comprising more than 95% of the monomers according to the Formula 1);
optionally submitting the coated beads to a sterilization treatment.

An alternative method to produce a 3D support for cell culture comprising the step of:
selecting or obtaining alginate beads;
submitting the alginate beads to a sterilization treatment
coating these beads with the polymer described here above (e.g. comprising more than 95% of the monomers according to the Formula 1), preferably by suspending the beads in a solution comprising the polymer described here above (comprising more than 95% of the monomers according to the Formula 1).

Preferably, in this method or in this alternative, the polymer for the coating is present in a composition at a concentration comprised between 0.01 and 1 mg/ml, preferably between 0.03 and 0.9 mg/ml, preferably between 0.05 and 0.8 mg/ml, preferably between 0.07 and 0.7 mg/ml, preferably between 0.1 and 0.5 mg/ml or between 0.15 and 0.5 mg/ml, such as between 0.2 and 0.3 mg/ml.

A sterilization treatment by autoclaving is preferred, such as at a temperature above 100° C., for instance of about 120° C., and a pressure higher than 100000 Pa, such as 150000 or even about 200000 Pa.

Then inventors have found that, in the alternative method, the alginate beads have a tendency to shrink. Instead of being a problem, the inventors do consider it to be advantageous since the final diameter (after the autoclaving treatment) is now comprised between 150 μm and 500 μm, preferably between 200 μm and 300 μm, which is better for cell culture.

### EXAMPLES

### Example 1

L929 fibroblasts have been seeded on TCPS (Tissue Culture polystyrene) or on PS multiwells (24 wells) adopting an initial cell density of 10 000 cells per cm². The cell culture has been realized in a DMEM culture medium enriched with Fetal Bovine Serum (FBS) (10%) buffered at pH 7.4 (commercial DMEM solutions). The cell culture has been conducted at 37° C. under 5% CO₂ atmosphere according to the standard operation conditions typically used for animal cell culture. The evolution of cell density and morphology (spreading behavior, shape) over time have been observed with a reverse optical microscopy under visible non polarized light.

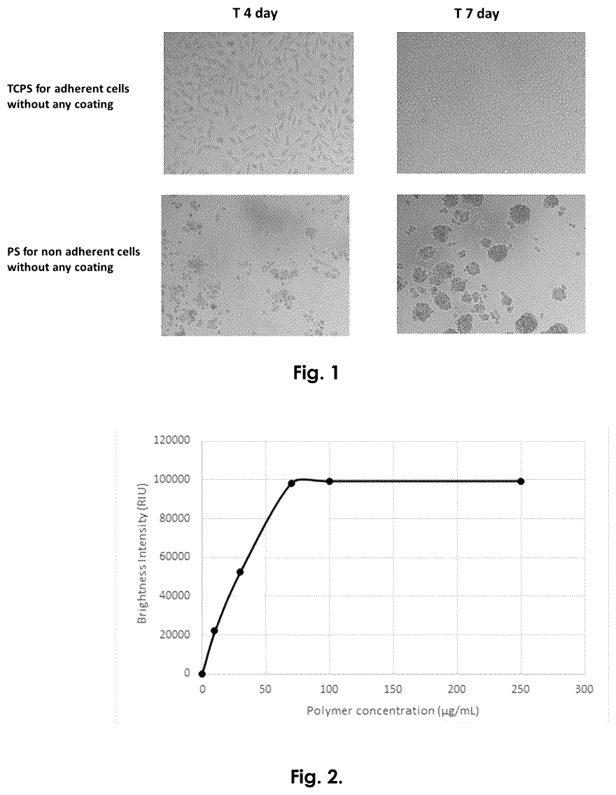

As reported on FIG. 1 the microscopic observations are highlighting that L929 cells cultured on TCPS are well spread after 4 days of culture while they appeared under the form of aggregates on non-functionalized PS. After 7 days of culture, fibroblasts are confluent and homogeneously distributed on TPCS, while they have generated big clusters on PS suitable for non-adherent cells.

### Example 2

PDMAEMA (10 kDa) has been prepared by living radical polymerization. After synthesis this polymer has been purified by 4 purification steps in order to eliminate any monomer, catalyst and solvent residue.

The efficiency of polycation deposition on TCPS has been determined using a fluorescent form of PDMAEMA (10 kDa) in function of the concentration of the polymer solution incubated on this solid substrate. 5 concentrations of PDMAEMA solution, ranging from 0.01; 0.03; 0.07; 0.1 to 0.25 mg/mL have been used for this coating. Epi-fluoresence microscopy has been used to verify the presence of the polymer on TCPS well but also to verify the homogeneity of the polymer coating. As noticed on FIG. 2, the mean fluorescent intensity measured from image analysis of the microscopic pictures taken using an epi-flurorescence microscope is highlighting a progressive increase in relative fluorescence intensity of the TCPS wells up to a polymer concentration in the solution incubated with the wells of 70 μg/mL. The microscopic images also highlight the homogeneity of fluorescence signal in the microscopic field which have been recorded.

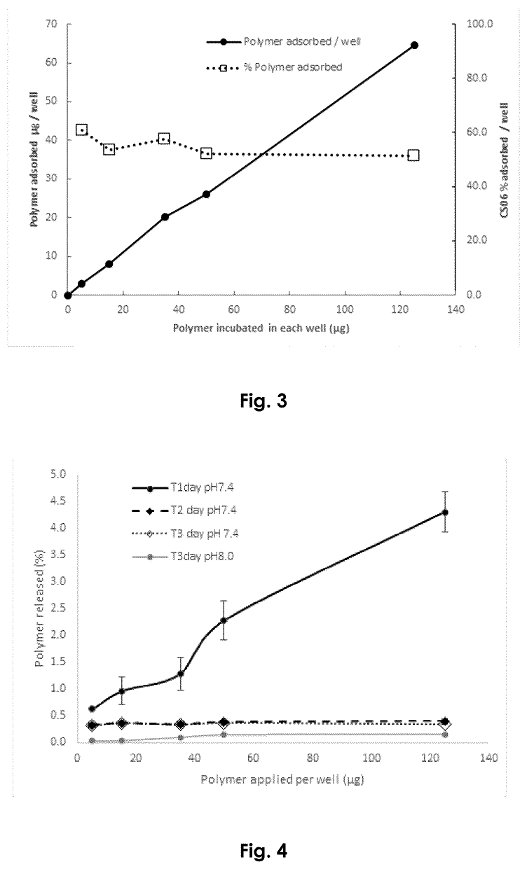

The fluorescent PDMAEMA coated on TCPS has been quantified measuring the fluorescence signal of the fluorescent PDMAEMA solutions recovered after their incubation on the TCPS multiwells for 1 h. The absolute amount of fluorescent PDMAEMA coated on TCPS has been measured by spectrofluorescence against a calibration curve established using well-known concentration of the fluorescent PDMAEMA. Interestingly and surprisingly enough the evolution of the polymer adsorbed per well (μg) is linear with the polymer amount added per well in within the entire domain of polymer concentration assessed in this study, i.e. from 10 to 250 μg/mL (FIG. 3). Accordingly, the percentage of polymer adsorbed per well is relatively constant and is really high, i.e. around 40% of the total amount of polymer incubated with the TCPS wells. Taking into account that the total area exhibited per well for the 24 multiwells used in this study is 1.9 cm², it means that the polymer surface density at the highest concentration of polymer solution used, i.e. 250 μg/mL is ˜35 μg/cm².

In order to evaluate the stability of the polymer coating in the DMEM medium complemented with FBS (10%) typically used for animal cell culture, the fluorescent polymer coated multiwells have been incubated up to 3 days at 37° C. under 5% CO₂ (normal conditions) in this medium. Fluorescence polymer released in the culture medium has been determined 1, 2 and 3 days after this incubation.

In spite of the very high polymer surface density noticed on TCPS, PDMAEMA seems relatively well attached to the polystyrene substrate. Indeed the % of PDMAEMA released from the multiwell in the culture medium supplemented by FBS (10%) does not exceed 4.5% at the highest polymer concentration used for the coating (i.e. 250 μg/mL). Moreover this polymer released is only noticed after the first day of incubation performed in the standard cell culture conditions of culture (37° C./5% CO₂). Afterwards the amount of fluorescence retrieved in the culture medium is close to the background. Interestingly enough also is the fact that the increase in pH to 8.0 of DMEM does not elicit anymore release of the polycation in the culture medium.

### Example 3

PDMAEMA solutions of 1 and 10 mg/mL have been prepared by dissolving the freeze-dried polymer in ultrapurified water. After dissolution the PDMAEMA solutions have been sterilized by filtration on 0.22 μm sterile filter within laminar flow.

PolyStyrene (TCPS) or non-modified polystyrene (PS) 24 Multiwell have been coated with the PDMAEMA solutions by physical impregnation. Briefly 0.5 mL of the PDMAEMA solutions have been incubated within the well for a duration of 30 min at room temperature under slight lateral agitation (500 rpm) under sterile conditions.

The solutions of PDMAEMA have been eliminated by aspiration and replaced by 0.5 mL of DMEM culture medium complemented with FBS (10%).

L929 fibroblasts have been seeded on each well by adding 0.5 mL of a L929 fibroblast cell suspension to achieve a cell density of 10.000 cells/cm².

Cell culture and cell observation have realized as reported in example 1.

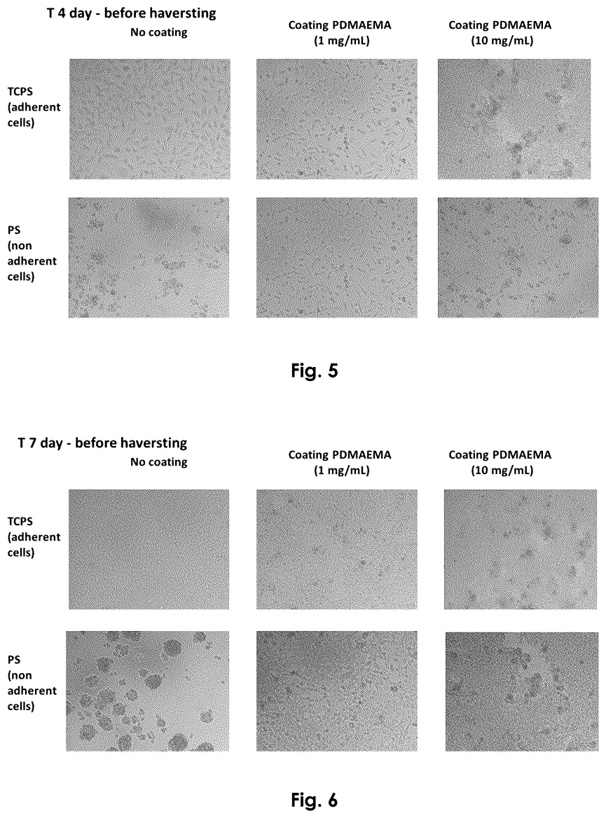

In contrast to the results outlined on example 1, the microscopic observations reported on FIGS. 5 and 6 are demonstrating that the simple physical coating of PDMAEMA (1 mg/mL) on polystyrene foreseen for non-adherent animal cells (PS) allows the adhesion and proliferation of fibroblasts L929. Indeed, very few cell aggregates are noticed under microscopic observation and the cell density looks similar compared to the control made of TCPS without any coating for day 4 and 7.

Interestingly enough, the same PDMAEMA coating (1 mg/mL) applied on polystyrene culture dishes made of TCPS does not interfere with the adhesion and proliferation of fibroblasts L929. Indeed, at day 4 the L929 cultivated on TCPS coated with PDMAMEA (1 mg/mL) are well spread with the typical elongation morphology. But more heterogeneities in cell shape are however noticed compared to the observation done on the control TPCS. The same observations have been done with PS coated with PDMAEMA (1 mg/mL). The inventors have noticed less heterogeneities at lower PDMAEMA concentration, and almost no heterogeneities at about 0.03 mg/ml.

In contrast when both types of polystyrene wells are coated with a higher concentration of PDMAEMA solution (i.e. 10 mg/mL), this coating significantly impacts the cell morphology and gives rise to cell aggregates already noticed at day 4 for both types of PS.

At day 7 the cell surface density is rather similar both for TCPS control, TCPS coated with PDMAMEA (1 mg/mL) or PS coated with PDMAEMA (1 mg/mL). But as noticed on day 4, the homogeneity of L929 spreading and shape is better for control TCPS. Those observations are therefore in favor of a similar proliferation rate whatever the surface of PS.

### Example 4

Suspecting a toxicity effect of PDMAEMA coated on TCPS at the highest concentration used last time (i.e. 10 mg/mL), while at 1 mg/mL most of the cells were alive, the inventors have assessed lower PDMAEMA concentration to coat TCPS, using one of the following polymer concentration: 0.1; 0.25 and 0.75 and 1 mg/mL.

PDMAEMA solutions at a concentration of 0.1; 0.25; 0.75 or 1 mg/mL have been prepared by dissolving a PDMAEMA of a mean molecular weight of 10 kDa under a freeze-dried form in ultrapurified water. The PDMAEMA solutions have been sterilized by filtration on 0.22 μm sterile filter within laminar flow.

PolyStyrene (TCPS) 24 Multiwell have been coated with the PDMAEMA solutions by physical impregnation according to the experimental conditions already reported on example 3.

L929 fibroblasts have been seeded on each well by adding 0.5 mL of a L929 fibroblast cell suspension to achieve a cell density of 10.000 cells/cm².

Cell culture and cell observation have been realized as reported in example 1.

As highlighted on FIG. 7, after 5 days of culture, the cell density is increasing when decreasing the concentration of PDMAEMA from 1 mg/mL to 0.1 mg/mL. When coated with a 0.1 mg/mL PDMAEMA solution no significant difference in cell density is noticed as compared to the control (without PDMAEMA) and in both cases cells are close to be confluent already.

At day 7 of culture, a higher density of cells is still observed at the lowest concentration of PDMAEMA, i.e. similar to the control, but the differences are lower. For the control and for PDMAEMA coating of 0.1 mg/mL, patches made from multilayers of cells can be visualized under microscopy.

### Example 5

Fibroblasts cultured on Tissue Culture PolyStyrene (TCPS) or non-modified polystyrene (PS) as described on example 3 have been detached after 7 days of culture.

This cell harvesting has been performed, first by elimination of the culture medium followed by the addition of 0.2 mL of:
either a classical protease enzymatic solution made of trypsin and EDTA solution;
or a DMEM culture medium, made from a commercial DMEM solution but whose pH has been increased to 8.0 with a sodium hydroxide solution (1M).

Cell detachment has been realized in these medium for 5 minutes under slight lateral agitation at 500 rpm at 37° C. under 5% CO₂ atmosphere.

After this period, 0.5 mL of DMEM culture medium containing 10% FBS have been added in each well.

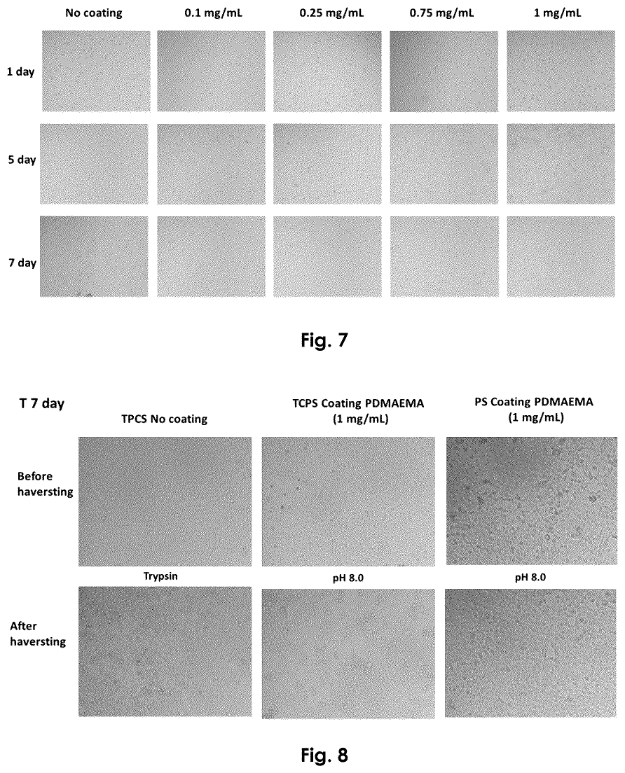

The detached cells have been observed under optical microscopy (FIG. 8).

After trypsin treatment, L929 cells are released from the TCPS surface. After being released from the TCPS surface, the fibroblasts have a spherical shape.

If TCPS has been previously coated with PDMAEMA (1 mg/mL), the slight alkalinization of DMEM to pH 8.0 for 5 min allows a significant harvesting of fibroblasts L929 which disclose also a spherical shape.

In contrast the alkalinization of the culture medium to pH 8.0 for 5 min does not affect cell adhesion cultured on native PS. Indeed, cells remain mostly attached and spread on TCPS coated with PDMAEMA after the alkalinization treatment.

Cells have been homogenized by 3 up-and-down aspirations with 1 mL pipette and transferred in Falcon tube of 15 mL before mixing under vortex stirrer, type Reax Top, VWR, fixed at maximum speed no 6.

0.2 ml of each suspension have been withdrawn and transferred in 96 multiwell plates made of TCPS.

In order to quantify cell detached from the multiwell and measure the viability of the cells, a MTT has been carried out according to the following way:
Cells have been incubated for 2 h at 37° C. under 5% CO₂ atmosphere to promote cell adhesion;
The supernatant of each well has been removed carefully by aspiration;
Cell viability and cell counting have been realized using the MTT assay. Briefly,
   0.2 mL of MTT reagent (0.5 mg MTT/ml of DMEM without FBS)
   Incubation for 2 h at 37° C. under 5% CO₂ atmosphere
   Removing of the supernatant carefully by aspiration
   Addition of 0.2 mL of DMSO
   Incubation of the multiwell for 2 h at room temperature under slight lateral agitation (500 rpm)
   The optical densities (DO) of the wells being part of the multiwell are measured at 540 nm using a commercial multiwell reader.
   The Dos are converted in terms of cell number per well adopting a calibration curve made using multiwells containing well-known amount of cells per well, i.e. ranging between 5,000 to 80,000 and incubated in the same conditions as reported above for the experimental assays.

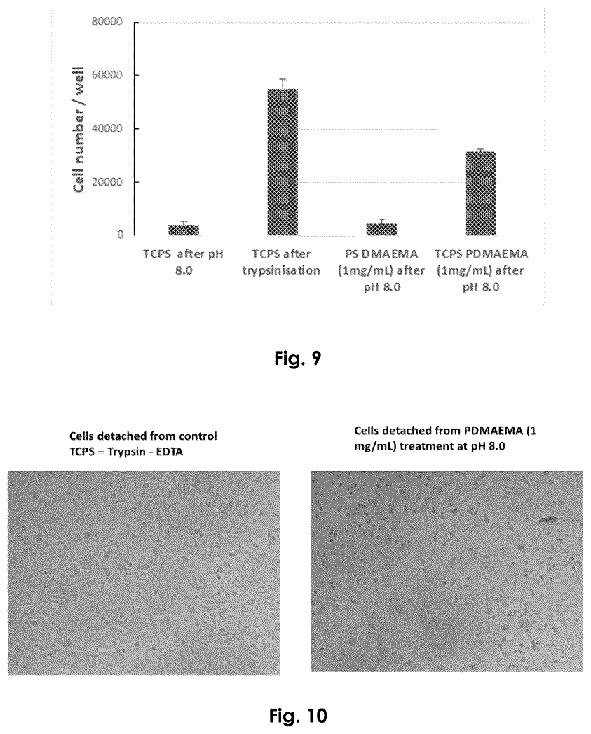

The MTT results (FIG. 9) allows to notice that the cell cultured on PS coated with PDMAEMA are not efficiently detached after the slight alkalinization of DMEM to pH 8.0 in contrast to what has been observed with TCPS coated with the same polymer. Accordingly, the combination of the results coming from microscopic observations and of MTT counting allows to conclude that this lower number of viable cells recovered from PS surface coated with PDMAEMA (1 mg/mL) is not arising from their low proliferation rate on this surface, but from their poor detachment PS surface coated with PDMAEMA (1 mg/mL).

This surprising difference in harvesting efficiency of L929 cultured on either TCPS or PS, both physically coated with the same polymer in the same experimental conditions is explained by the inventors by the fact that TCPS is bearing anionic groups (sulfonate), while PS foreseen for the culture of non-adherent cells should be hydrophobic and neutral. According to these differences in surface chemistry between TCPS and PS, the inventors consider a difference in adsorption behavior between PDMAEMA and these two polymeric surfaces. In the case of TCPS the inventors expect that ionic interactions prevail, while polar or van der Waals interactions forces mostly promote the anchorage of PDMAEMA on non PS.

Accordingly, the inventors have shown that a slight increase of the pH of DMEM culture medium to 8.0, i.e. sufficient to mostly neutralize the ternary amine of PDMAEMA, is enough to promote cell detachment from the surface, which have adhered originally thanks to the cationic group of this polycation physically coated on the TCPS surface.

In the case of non-functionalized PS, the inventors anticipate that polar forces between PDMAEMA and PS should not be influenced by the pH, therefore explaining why fibroblasts have remained attached on PS after the slight alkalinization to pH 8.0.

In addition to the demonstration of the viability of L929 after their culture on TCPS coated on PDMAEMA (1 mg/mL) and their harvesting by a slight alkalinization at pH 8.0 for 5 min, the inventors have also verified their ability to adhere and spread quickly after their seeding on TCPS. As disclosed on FIG. 10, no significant difference in cell phenotype behavior between fibroblasts either detached by trypsin from control TCPS or either detached from PDMAEMA coated TCPS.

### Example 6

Fibroblasts cultured on Tissue Culture PolyStyrene (TCPS) coated with PDMAEMA at a concentration of 0; 0.1; 0.25; 0.75 or 1 mg/mL as described on example 4 have been detached after 5 or 7 days of culture.

This cell harvesting has been performed, first by elimination of the culture medium followed by the addition of a trypsin solution or an incubation at pH 8.0.

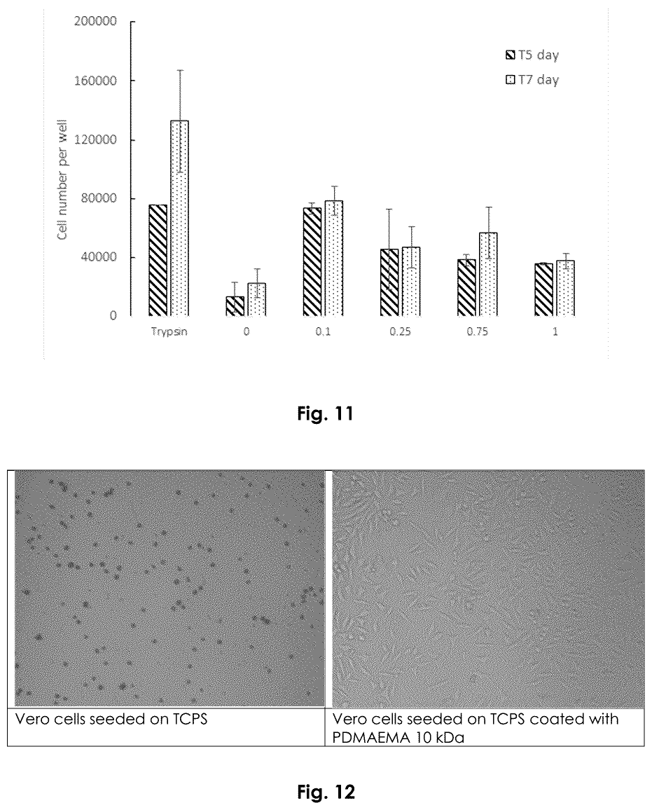

The results given on FIG. 11 is highlighting that:
At day 5 day of culture:

No significant cell detachment is noticed for TCPS without PDMAEMA coating after incubation at pH 8.0.

A similar cell detachment efficiency and cell viability is noticed between the trypsin control and the TCPS wells coated with the lowest concentration of PDMAEMA (0.1 mg/mL).

Above this polymer concentration, the amount of cells recovered after the slight alkalinization is decreasing, thus in agreement with the difference in cell density noticed under microscopical observations outlined on FIG. 7.
At day 7 day of culture:

A similar amount of cells are recovered from wells coated with PDMAEMA compared to day 5, i.e. with a decrease in cell number with the concentration of PDMAEMA solution used to perform the coating.

The cell recovery for the trypsin control is 60% higher, as compared to the wells coated with the PDMAEMA: the higher cell density reduces the effect of the alkalinization.

To sum up, the inventors consider that the adhesion, proliferation rate, shape, viability and detachment efficiency of fibroblasts cultivated on PDMAEMA coated TCPS are similar compared to control TCPS if concentration of the PDMAEMA solution used to coat the substrate is below 0.2 mg/mL and if cells are not cultivated above confluency.

### Example 7

Vero cells are known to recover very slowly after freezing. Therefore, several weeks are known to be necessary to recover them before proceeding to their first passage on a new TCPS surface. In order to enhance their in vitro recovery, adhesion, spreading and proliferation, the inventors have seeded them after thawing on TCPS first coated with PDMAEMA.

As outlined on example 2 PolyStyrene (TCPS) 24 Multiwells have been coated with the PDMAEMA solutions by physical impregnation. Briefly 0.5 mL of the PDMAEMA solutions has been incubated within the well for a duration of 30 min at room temperature under slight lateral agitation (500 rpm) under sterile conditions.

The solutions of PDMAEMA have been eliminated by aspiration and replaced by 0.5 mL of DMEM culture medium complemented with FBS (10%).

1 mL of Vero cell suspension, i.e. cell line isolated from kidney epithelial cells extracted from an African green monkey, stored in liquid nitrogen, has been quickly thawed at 37° C. and diluted afterwards in 9 mL of DMEM containing 15% FBS. This cell suspension has been centrifuged for 5 min at 200 g in order to eliminate DMSO. The supernatant has been discarded by aspiration and the cell pellet has been suspended in 3 mL of DMEM containing 15% FBS by vortexing for 10 sec. 1.5 mL of this cell suspension has been transferred in one well of PolyStyrene (TCPS) 12 Multiwells coated with PDMAEMA. As a control, 1.5 mL of the same cell suspension has been transferred in one well of PolyStyrene (TCPS) 12 Multiwells without PDMAEMA coating.

Cell culture and cell observation have been realized as reported in example 1. As highlighted on FIG. 12, Vero cells seeded on PDMAEMA-coated TCPS have quickly adhered, spread and proliferated on this surface after 1 week. In contrast the same cells cultured on non-coated TCPS have not adhered at all, disclosing spherical shape after the same period of incubation.

### Example 8

Dissolvable alginate microcarriers have been adopted as a 3D substrate for animal cell amplification. Alginate is non-toxic for animal cells and is safe for human being including for parenteral purposes. However due to its high negative charge density at neutral pH, animal cells do not adhere to the surface of this polymer. With the purpose to improve cellular adhesion on alginate microcarriers the inventors have coated on their surface the same polymers and the same coating procedure mentioned in example 2.

Alginate microcarriers have been realized in order to promote animal cell adhesion and proliferation. The inventors do consider that the adoption of these innovative microcarriers to amplify animal cells, especially stem cells, offer as main advantages to increase the total surface available for cell adhesion and proliferation compared to 2D substrates. Moreover the inventors have found an additional benefit of the alginate microcarriers performed as described below: their quantitative and rapid dissolution ability under well-controlled and safe conditions to guarantee the viability and functionality of animal cells. Accordingly, upon cell amplification on these functionalized microcarriers and after their dissolution, the harvested cells can be easily recovered, but also purified, by simple centrifugation and washings to eliminate the dissolved alginate and the coating polymer.

Alginate microcarriers can be easily prepared by dissolving sodium alginate in an aqueous solution. If several viscosity range of alginate can be used, such as from 20-50 mPa·s, to 100-200 mPa·s, 500-600 mPa·s (1%; 20° C.), an optimal viscosity ranges between 500 to 600 mPa·s to facilitate polymer injection and hardening. In practise 10 mL of a 0.5% alginate aqueous solution is injected at a flow-rate of 0.2 mL/min within a 2% calcium chloride solution through a 27 G needle inserted within a venturi system to produce spherical micro-droplets. The mean size of these droplets can be mostly adjusted and controlled by varying the geometry of the venturi device and by the pressure and flow-rate of air pressure co-injected around the extremity of the 27 G needle. Upon solidification, calcium alginate particles are washed with water before proceeding to their polycation coating.

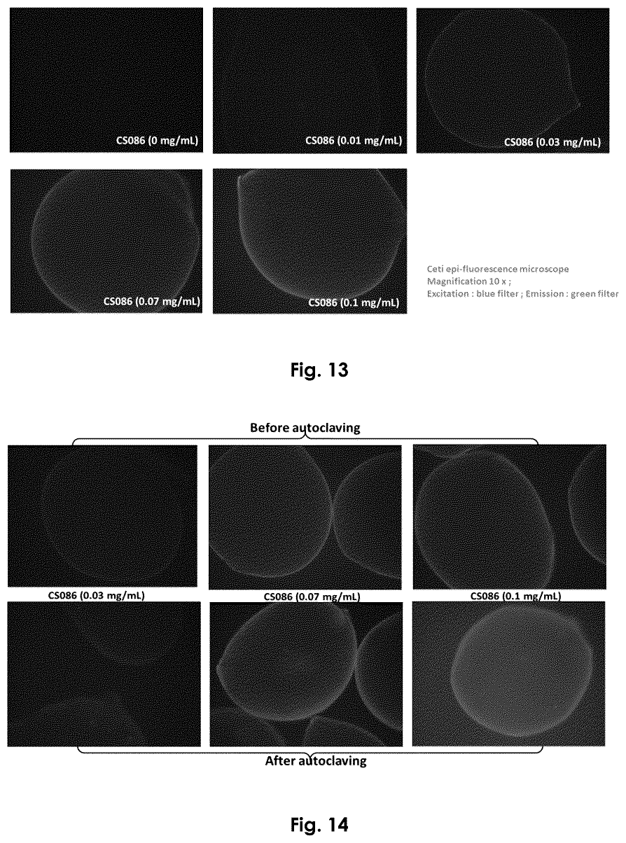

The surface of the alginate microcarriers have been coated by suspending them in a solution of the polymer listed in example 1 and dissolved in an aqueous medium to achieve final concentration ranging between 0.01 to 0.1 mg/mL. In view to visualize the polymer coating and its stability, a fluorescent polycation labelled with fluorescein has been adopted. Upon incubation under rolling agitation, the excess of polycation has been eliminated by 3 washing cycles performed in ultrapure water. As highlighted on FIG. 13, after coating with the fluorescent polymer, the alginate microcarriers are clearly evidenced under epi-fluorescence microscopy using a blue filter for excitation and a green filter for emission. A clear dependence is also noticed between the fluorescent signal given by the alginate microcarriers and the concentration of the polycation solution used to coat them. This fluorescence signal is homogeneously distributed on the surface of the microcarriers.

Upon coating the alginate microcarriers can be sterilized either by autoclaving at 121° C. under pressure according to standard operation procedure, either by incubation in ethanol 100% for 15 min before washing them in a sterile aqueous medium. For industrial purposes, the coated microcarriers can also be dried and/or sterilized by beta or gamma radiations or by ethylene oxide treatment following standard conditions applied in the medical and pharmaceutical industry.

As evidenced on FIG. 14, the autoclaving cycle does not impair the stability of the alginate microcarriers. Their morphology has not significantly changed and microparticles are not aggregated. Interestingly their fluorescence coating is still well present around the microcarriers and is slightly enhanced after this sterilisation process.

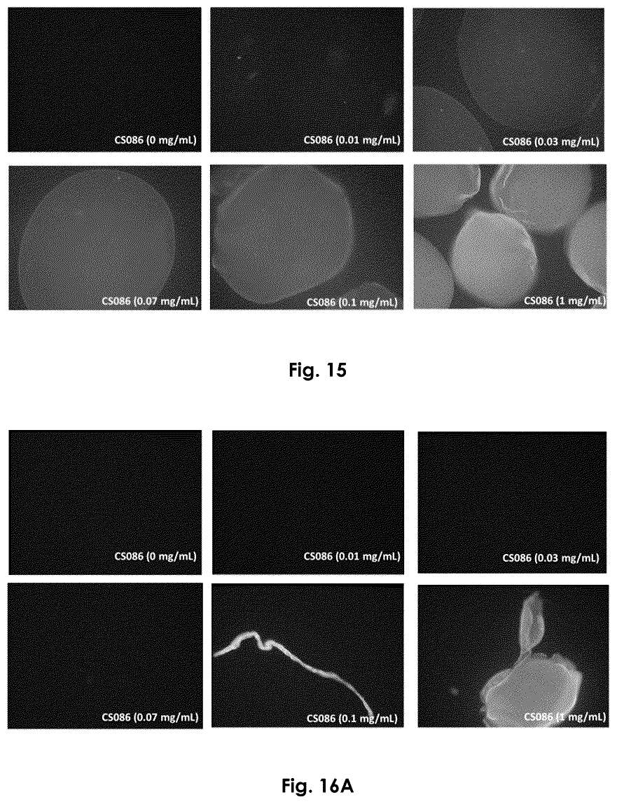

The physico-chemical stability of the polymer coating has been also assessed after incubation of alginate microcarriers coated with raising concentrations (0.01 to 1 mg/mL) of a fluorescent PDMAEMA. After incubation for 1 day in NaCl (9 g/L), and 3 days in DMEM culture medium supplemented with FBS (10%) at 37° C., the epi-fluorescence observations are demonstrating that the fluorescent polymer remains well anchored to the surface of the microcarriers (FIG. 15). The alginate microcarriers remains also stable in these conditions. Interestingly enough, without polymer coating the alginate microcarriers have been totally dissolved after their incubation in the isotonic sodium chloride solution (9 g/L) carried out overnight at room temperature.

Although stable against these different medium and temperature/pressure stresses, dissolution of the coated alginate microcarriers has been successful after their incubation within a physiological phosphate buffer saline (PBS) for 90 min at 22° C. As reported on FIGS. 16A (fluorescent coating) and B (normal light; alginate core), this dissolution is complete at least for microcarriers coated with polycation solution concentration up to 0.07 mg/mL. At higher polycation concentration used for microcarrier coating, the microcarriers are well damaged, but polymeric fragments are still visible under fluorescent or normal light transmission microscopy, especially at 1 mg/mL (FIGS. 16A and B).

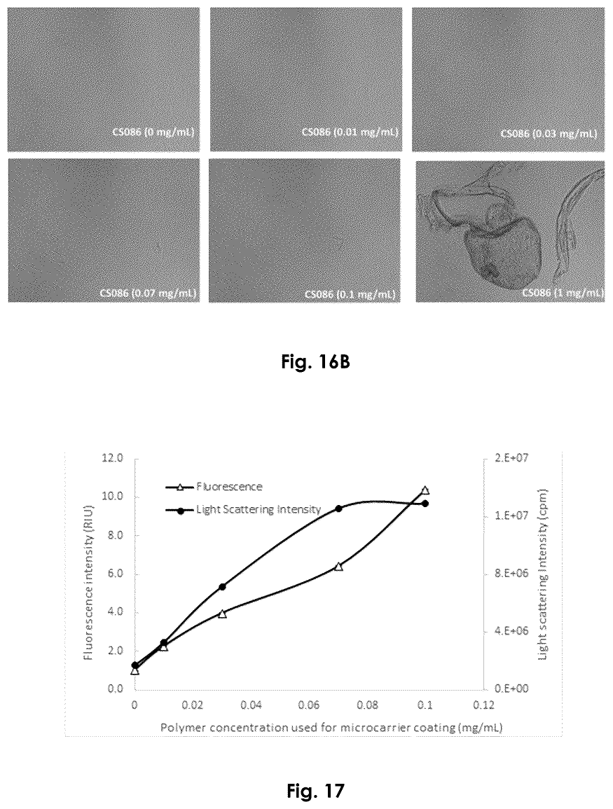

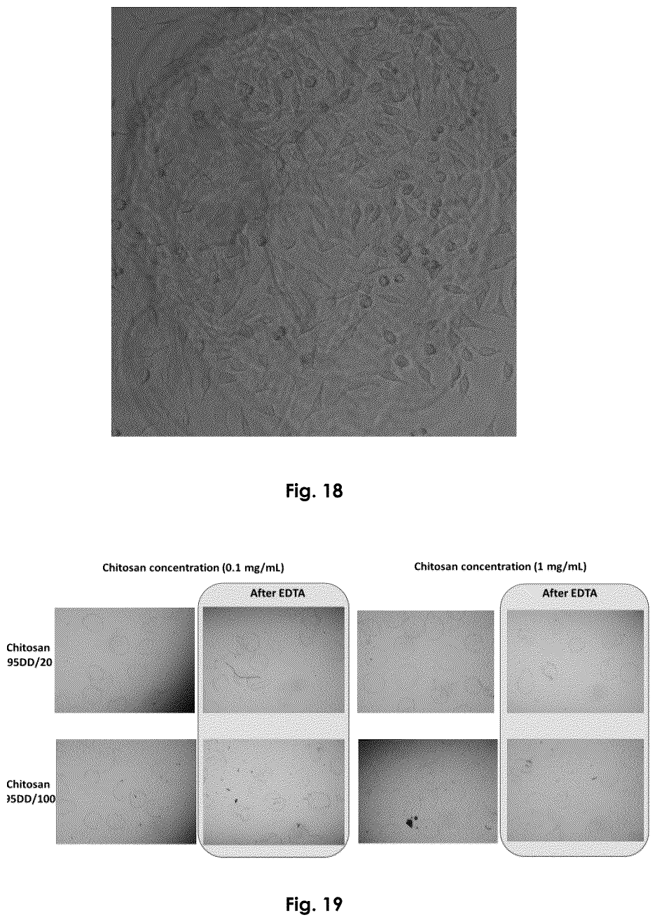

An alternative methodology found to dissolve more quickly and efficiently the coated alginate microcarriers relies on their incubation within a more potent chelator than phosphate anions. Most specifically the alginate microcarriers coated with fluorescent polycation solution raising in concentration up to 1 mg/mL have been efficiently dissolved after their incubation for 15 min in an isotonic sodium EDTA solution buffered at pH 7.4, giving rise to transparent solutions. This dissolution process has been first verified macro- and microscopically and has been quantified by Dynamic Light Scattering (DLS) and fluorescent spectrometry of the resulting solution. As noticed on FIG. 17, these two parameters are increasing in close correlation with the fluorescent polycation concentration adopted to coat the microcarrier, thereby supporting the entire dissolution of the alginate core, but also of the polycation shell of the microcarriers.

### Example 9

Fibroblasts, L929, have been cultivated at the surface of alginate microcarriers coated with PDMAEMA (10 Kda) in order to verify their cell adhesion ability. In practice, the coated alginate microcarriers have been first incubated for 30 min in the cell culture medium DMEM, containing 10% of fetal bovine serum (FBS).

0.1 mL of alginate microcarriers has been placed within well of a 24 multiwell plate in order to have a covering the total area of the well. L929 cells have been seeded afterwards by adding 1 mL of the cell suspension in view to reach 40.000 cells within each well. The cells combined with the microcarriers have been incubated at 37° C. within a CO₂ incubator and placed under lateral agitation according to the following agitation cycle:
Agitation just after seeding for 5 min at 300 rpm
agitation for 1 min à 300 rpm every 15 min up to 2 h

Cell incubation has been continued afterwards without any more agitation for 3 days at 37° C. under 5% CO₂. As disclosed on FIG. 20, fibroblasts have been well adhered, spread and proliferate to the surface of the PDMAEMA coated alginate microcarriers.

### Example 10

Coating of Alginate Microcarriers with Chitosan.

If other polycations could be adsorbed to the surface of alginate microcarriers, the resulting polyelectrolyte complex, which could be realized at the particle surface does not obviously satisfy to the three main expected functionalities of the microcarriers described in this patent, i.e. cell adhesion properties, cell safety and ability to dissolve quickly upon request, i.e. after cell amplification. Since years the literature has disclosed that chitosan, a natural well-known polycation, could indeed adsorb to the surface of alginate microcarriers. But this coating has been used in view to form stable capsules which can resist in a physiological environment for months due to the very high stability of the polyelectrolyte film generated by the interaction of alginate and chitosan.

In order to compare precisely the behaviour of chitosan to the polymers referred above, the inventors have proceeded to a coating of the same autoclaved alginate microcarriers reported in example 9, but using 2 chitosan molecules differing in terms of molecular weights. Accordingly, their dynamic viscosity are equal respectively to 16-30 mPas and 71-150 mPas (1% in 1% acetic acid, 20° C.) for chitosan labelled 95DD/20 and 95DD/100.

These chitosan molecules have been dissolved at 3 concentrations (0.03; 0.1 and 1 mg/mL), in order to coat alginate microcarriers in view to cover the same concentration range used with PDMAEMA. The microcarrier coating has been performed according to the same experimental conditions reported in example 9 with PDMAEMA exception made that the lack of solubility of chitosan at neutral pH has imposed to realize the coating in a medium buffered at pH 4.5 to keep this polymer in solution. At the lowest chitosan concentrations used (i.e. 0.03 mg/mL) the alginate microcarriers have been totally dissolved upon their incubation with chitosan whatever its molecular weight. At the two higher chitosan concentrations (0.1 and 1 mg/mL) the alginate microcarriers have mostly maintain their spherical shape as highlighted on FIG. 19.

However and in opposite to the same alginate microcarriers coated with the polymers referred above, upon treatment in EDTA (5 mM) and whatever the duration of incubation, the chitosan coated particles have resisted to this calcium chelation process.

It should be understood that the present invention is not limited to the described embodiments and that variations can be applied without going outside of the scope of the claims.

1. A method to grow animal cells, preferably mammalian cells and/or stem cells, comprising the steps of:
selecting a surface,
coating the said surface with a polymer substantially consisting of a succession of monomers according to the formula I 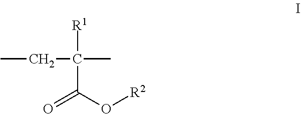
and
growing the said animal (mammalian and/or stem) cells on the said coated surface for a period of time so that the said animal (mammalian and/or stem) cells adhere to the said coated surface,
wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group;
wherein the said polymer has a mean pKa between 6.0 and less than 7.5 and a charge density at pH 7.0 between 20 and 50% (number of the positively charged monomers:total number of monomers).

2. The method according to claim 1, wherein the surface is negatively charged, preferably wherein the surface is selected from the group of plasma-treated polystyrene, glass and alginate.

3. The method of claim 1, further comprising the step of detaching the grown animal cells upon increasing the pH of the medium to a pH comprised between 7.8 and 8.2 and/or upon adding a compound selected from a calcium chelator, such as EDTA or EGTA or citrate or phosphate anions, and low molecular weight cations, such as betaine, spermidine or spermine.

4. The method according to claim 1, wherein the culture medium and/or the detachment medium comprises at least 5 mM phosphate, preferably at least 10 mM phosphate, the said detachment medium being substantially devoid of calcium.

5. The method according to claim 1, wherein the polymer is comprising more than 80%, preferably more than 90% and more preferably more than 95% of the monomers according to the formula I, such as between 95% and 99% (number of the monomers according to the formula I:total number of the monomers), and/or wherein R¹ is a methyl, and/or wherein the polymer harbors a charge density at pH 7.0 between 25 and 40%, preferably between 30 and 35% (number of the positively charged monomers:total number of monomers), and/or harbors less than 10% of negative charge density at pH 7.0, preferably less than 5%, more preferably less than 2% (number of the negatively charged monomers:total number of monomers).

6. The method according to claim 1, wherein the polymer is coated on the surface upon incubation at a concentration between 0.001 mg/ml and 1.0 mg/ml, preferably 0.002 mg/ml and 0.5 mg/ml, more preferably between 0.002 mg/ml and 0.3 mg/ml.

7. The method according to claim 1, wherein the polymer is according to the formula II 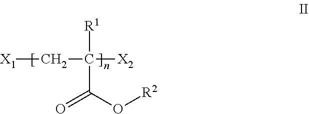
wherein X₁ and X₂ represent the alpha and omega end groups of the said polymer; wherein the said X₁ and X₂ independently represent a hydrogen atom, a hydroxyl group, an ethyl isobutyrate group, an alkyl group, a halogen group, a carboxylic acid group, an amino group, a methoxy group or an ethoxy group and wherein n is the number of the repetitive monomer units according to the formula I.

8. A composition comprising a surface coated with a polymer substantially consisting of a succession of monomers according to the formula I 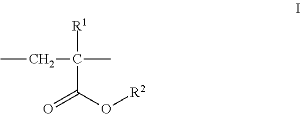
wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group;
preferably, wherein the said polymer has a mean pKa between 6.0 and 7.5 and/or a charge density at pH 7.0 between 20 and 50% (number of the positively charged monomers:total number of monomers).

9. The composition of claim 8, wherein the surface is negatively charged, preferably wherein the surface is selected from the group of plasma-treated polystyrene, glass and alginate.

10. The composition according to claim 8, wherein the polymer is comprising more than 80%, preferably more than 90% and more preferably more than 95% of the monomers according to the formula I, such as between 95% and 99% (number of the monomers according to the formula I:total number of the monomers), and/or wherein R¹ is a methyl group.

11. The composition according to claim 8, wherein the polymer harbors a charge density at pH 7.0 between 25 and 40%, preferably between 30 and 35% (number of the positively charged monomers:total number of monomers), and/or harbors less than 10% of negative charge density at pH 7.0, preferably less than 5%, more preferably less than 2% (number of the negatively charged monomers:total number of monomers).

12. The composition according to claim 8, wherein the polymer is coated on the surface upon incubation at a concentration between 0.001 mg/ml and 1.0 mg/ml, preferably 0.002 mg/ml and 0.5 mg/ml, more preferably between 0.002 mg/ml and 0.3 mg/ml.

13. The composition according to claim 8 further comprising animal cells, preferably mammalian cells and/or stem cells adhered on the polymer.

14. The composition of claim 13, wherein the animal and/or mammalian and/or stem cells are adhered or inoculated at a known density.

15. The composition according to claim 8 wherein the coated surface is in the form of particles, preferably in suspension in a cell culture medium.

16. Use of the composition according to claim 8 or of a polymer substantially consisting of a succession of monomers according to the formula I 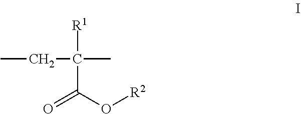
wherein R¹ represents a hydrogen atom or a straight or branched chain alkyl group from 1 to 6 carbon atoms;
wherein R² represents a straight or branched chain alkyl group from 1 to 6 carbon atoms, which is substituted by a protonable amino group;
preferably, wherein the said polymer has a mean pKa between 6.0 and 7.5 and/or a charge density at pH 7.0 between 20 and 50% (number of the positively charged monomers:total number of monomers)
for an in vitro culture of animal cells, preferably of mammalian cells and/or of stem cells.

17. A pharmaceutical composition comprising the animal (mammalian and/or stem) cells or parts thereof or the secretion products thereof, obtainable by the method according to claim 1.

18. A method to produce a 3D support for cell culture comprising the step of:
selecting or obtaining alginate beads;
optionally submitting the said alginate beads to a sterilization treatment
coating the said beads with a polymer substantially consisting of a succession of monomers according to the formula I

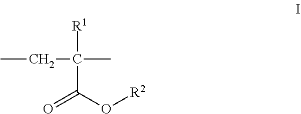

19. The method of claim 18, wherein the coating step is performed upon suspending the alginate beads in a solution comprising the polymer of claim 18, wherein the said polymer is at a concentration ranging between 0.01 and 1 mg/ml.

20. The method of claim 18, wherein the sterilization treatment is autoclaving at a temperature above 100° C. and a pressure above 100000 Pa.
